(19) United States
(12) Patent Application Publication
(10) Pub. No.: US 2019/0210951 A1
(43) Pub. Date: Jul. 11, 2019

(54) METHOD OF HYDROGENATION

(71) Applicant: ROHM AND HAAS COMPANY, COLLEGEVILLE, PA (US)

(72) Inventors: JOSE ANTONIO TREJO O'REILLY, Collegeville, PA (US); Alfred K SCHULTZ, Collegeville, PA (US)

(21) Appl. No.: 16/325,791

(22) PCT Filed: Sep. 6, 2017
(86) PCT No.: PCT/US2017/050179

§ 371 Date: Feb. 15, 2019

Related U.S. Application Data
(60) Provisional application No. 62/396,928, filed on Sep. 20, 2016.

(51) Int. Cl. C07C 45/00; B01J 31/08; B01J 23/44

(52) U.S. Cl. CPC C07C 45/006; B01J 23/44; B01J 31/08

(57) ABSTRACT

Provided is a method of hydrogenation comprising forming a reaction mixture comprising
(a) one or more reactant selected from the group consisting of phenol, one or more derivatives of phenol, and mixtures thereof;
(b) hydrogen; and
(c) catalyst, wherein the catalyst comprises beads that comprise one or more acid-functional organic resin and one or more metal selected from the group consisting of palladium, platinum, silver, gold, rhodium, ruthenium, copper, iridium, and mixtures thereof.

A hydrogenation reaction that is often desired is the conversion of phenol or a derivative of phenol to cyclohexanone or to a derivative of cyclohexanone. Such hydrogenations are sometimes performed by bringing phenol or a derivative of phenol into contact with a catalyst. WO 2015163221 describes a hydrogenation process involving contact between phenol and a catalyst, and the catalyst described by WO 2015163221 contains metal and has a carrier such as silica, alumina, silica-alumina, zirconia, zeolites, or activated carbon.

It is desired to provide a method of hydrogenation that uses a metal-containing catalyst that has a carrier that is an organic resin. It is contemplated that such a catalyst would have one or more of the following advantages: capability of performing catalysis at relatively low temperature; good resistance to leaching out of metal loaded onto the catalyst; good mechanical stability; and relatively high concentration of metal.

The following is a statement of the invention.

A first aspect of the present invention is a method of hydrogenation comprising forming a reaction mixture comprising
(a) one or more reactant selected from the group consisting of phenol, one or more derivatives of phenol, and mixtures thereof;
(b) hydrogen; and
(c) catalyst, wherein the catalyst comprises beads that comprise one or more acid-functional organic resin and one or more metal selected from the group consisting of palladium, platinum, silver, gold, rhodium, ruthenium, copper, iridium, and mixtures thereof.

The following is a detailed description of the invention.

As used herein, the following terms have the designated definitions, unless the context clearly indicates otherwise.

As used herein, hydrogenation is a chemical reaction in which an initial compound that contains a carbon-carbon double bond reacts so that the carbon-carbon double bond becomes a carbon-carbon single bond, and each carbon in the bond becomes bonded to a new hydrogen atom that was not present in the initial compound. As used herein, the term “hydrogenation” applies to such chemical reactions when the carbon-carbon double bond in the initial compound is either an aromatic double bond or an aliphatic double bond.

Phenol and derivatives of phenol have structure (I):

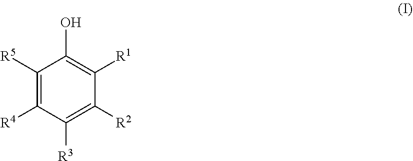

where each of R¹, R², R³, R⁴, and R⁵ is hydrogen or an organic group. When each of R¹, R², R³, R⁴, and R⁵ is hydrogen, the compound is phenol. Cyclohexanone and derivatives of cyclohexanone have the structure (II):

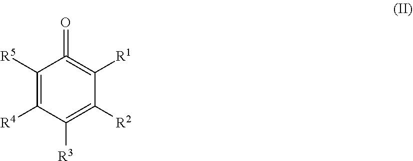

where R¹, R², R³, R⁴, and R⁵ are defined as in structure (I). When R¹, R², R³, R⁴, and R⁵ are each hydrogen, then the compound is cyclohexanone.

As used herein, “beads” are particles of material that are solid at 25° C. A bead that is not spherical is considered to have a diameter that is the same as the diameter of a sphere having the same volume as the non-spherical bead. A collection of beads is characterized by the harmonic mean diameter of the collection.

“Resin” as used herein is a synonym for “polymer.” A “polymer,” as used herein is a relatively large molecule made up of the reaction products of smaller chemical repeat units. Polymers may have structures that are linear, branched, star shaped, looped, hyperbranched, crosslinked, or a combination thereof; polymers may have a single type of repeat unit (“homopolymers”) or they may have more than one type of repeat unit (“copolymers”). Copolymers may have the various types of repeat units arranged randomly, in sequence, in blocks, in other arrangements, or in any mixture or combination thereof. Polymers have weight-average molecular weight of 2,000 or more.

Molecules that can react with each other to form the repeat units of a polymer are known herein as “monomers.” The repeat units so formed are known herein as “polymerized units” of the monomer.

Organic polymers are polymers selected from vinyl polymers, polyethers, polyamides, polyesters, phenol-formaldehyde polymers, polyurethanes, epoxies, polydienes, and mixtures thereof.

Vinyl monomers have a non-aromatic carbon-carbon double bond that is capable of participating in a free-radical polymerization process. Vinyl monomers have molecular weight of less than 2,000. Vinyl monomers include, for example, styrene, substituted styrenes, dienes, ethylene, ethylene derivatives, and mixtures thereof. Ethylene derivatives include, for example, unsubstituted and substituted versions of the following: vinyl acetate and acrylic monomers. “Substituted” means having at least one attached chemical group such as, for example, alkyl group, alkenyl group, vinyl group, hydroxyl group, alkoxy group, hydroxyalkyl group, carboxylic acid group, sulfonic acid group, quaternary ammonium group, other functional groups, and combinations thereof.

Monofunctional vinyl monomers have exactly one polymerizable carbon-carbon double bond per molecule. Multifunctional vinyl monomers have two or more polymerizable carbon-carbon double bonds per molecule.

As used herein, acrylic monomers include acrylic acid, methacrylic acid, esters thereof, amides thereof, acrylonitrile, and methacrylonitrile. Esters of acrylic acid and methacrylic acid include alkyl esters in which the alkyl group is substituted or unsubstituted. Amides of acrylic acid and methacrylic acid include amides in which the nitrogen atom of the amide group is either substituted or unsubstituted.

As used herein, vinyl aromatic monomers are vinyl monomers that contain one or more aromatic ring.

Vinyl monomers are considered to form polymers through a process of vinyl polymerization, in which the carbon-carbon double bonds react with each other to form a polymer chain.

A polymer in which 90% or more of the polymerized units, by weight based on the weight of the polymer, are polymerized units of one or more vinyl monomers is a vinyl polymer. A vinyl aromatic polymer is a polymer in which 50% or more of the polymerized units, by weight based on the weight of the polymer, are polymerized units of one or more vinyl aromatic monomer. A vinyl aromatic polymer that has been subjected to one or more chemical reactions that result in acid-functional groups being attached to the vinyl aromatic polymer is still considered herein to be a vinyl aromatic polymer. An acrylic polymer is a polymer in which 50% or more of the polymerized units, by weight based on the weight of the polymer, are polymerized units of one or more acrylic monomer. An acrylic polymer that has been subjected to one or more chemical reactions that result in acid-functional groups being attached to the acrylic polymer is still considered herein to be an acrylic polymer.

A resin is considered herein to be crosslinked if the polymer chain has sufficient branch points to render the polymer not soluble in any solvent. When it is said herein that a polymer is not soluble in a solvent, it means that less than 0.1 gram of the resin will dissolve in 100 grams of the solvent at 25° C.

A resin is considered acid-functional when acid-functional groups are covalently bound to the resin. The acid functional groups may be covalently bound directly to an atom in the main chain of the polymer, or the acid groups may be covalently bound to an intermediate chemical group that is, in turn covalently bound to an atom in the main chain of the polymer, or a combination thereof. Acid-functional groups include carboxylic acid groups, sulfonic acid groups, phosphorous-containing acid groups, and mixtures thereof. The term “acid-functional groups” includes both the protonated form of the group and the anionic form of the group.

A resin is considered crystalline if it shows a melting peak when analyzed by differential scanning calorimetry (DSC) at 10° C./min. A melting peak is an endotherm, and the area of the melting peak is related to the percentage of the resin that is crystalline and to the heat of fusion of the resin. A resin that does not show an appreciable melting peak in DSC is considered amorphous.

Ratios presented herein may be characterized as follows. For example, if a ratio is said to be 3:1 or greater, that ratio may be 3:1 or 5:1 or 100:1 but may not be 2:1. For another example, if a ratio is said to be 15:1 or less, that ratio may be 15:1 or 10:1 or 0.1:1 but may not be 20:1. This characterization may be stated in general terms as follows. When a ratio is said herein to be X:1 or greater, it is meant that the ratio is Y:1, where Y is greater than or equal to X. Similarly, when a ratio is said herein to be W:1 or less, it is meant that the ratio is Z:1, where Z is less than or equal to W.

The reaction mixture of the present invention includes reactant (a), which is selected from phenol, one or more derivatives of phenol, and mixtures thereof. Phenol and its derivatives are defined by structure (I) above. R¹ through R⁵ may be different from each other, or two or more of R¹ through R⁵ may be the same as each other. Two or more of R¹ through R⁵ may be joined together to form a cyclic structure. Preferably, each of R¹ through R⁵ has 10 or fewer non-hydrogen atoms. Preferably, each of R¹ through R⁵ is independently hydrogen, hydroxyl, oxyalkyl, substituted alkyl, or unsubstituted alkyl. Among substituted alkyl groups, preferred are those in which the substituents include hydroxyl groups, alkoxy groups, or a combination thereof. More preferably, each of R¹ through R⁵ is independently hydrogen or unsubstituted alkyl. More preferably, each of R¹ through R⁵ is hydrogen.

Preferably, the hydrogenation reaction of the present invention produces cyclohexanone or a derivative thereof, as depicted above in structure (II). The suitable and preferred embodiments of R¹ through R⁵ are the same for cyclohexanone and its derivatives as the suitable and preferred embodiments of R¹ through R⁵ as described above for phenol and its derivatives. Preferably, R¹ on cyclohexanone or its derivative is the same as R¹ on phenol or its derivative. Preferably, R² on cyclohexanone or its derivative is the same as R² on phenol or its derivative. Preferably, R³ on cyclohexanone or its derivative is the same as R³ on phenol or its derivative. Preferably, R⁴ on cyclohexanone or its derivative is the same as R¹ on phenol or its derivative. Preferably, R⁵ on cyclohexanone or its derivative is the same as R⁵ on phenol or its derivative.

The beads used in the present invention comprise one or more acid-functional organic resin. Preferred acid-functional organic resins are vinyl polymers. More preferred are vinyl aromatic polymers and acrylic polymers. Preferred acid-functional groups are carboxylic acid groups and sulfonic acid groups. Two preferred types of acid-functional organic resins are as follows: resins (i), which are vinyl organic resins having sulfonic acid groups, and resins (ii), which are acrylic resins having carboxylic acid groups. More preferred are resins (ii), which are acrylic resins having carboxylic acid groups.

The acid-functional resin may be made by any method. In a preferred method, beads containing a preliminary copolymer are made by a process of aqueous suspension polymerization of a monomer mixture. Preferably, the preliminary copolymer has no acid-functional groups, and the preliminary copolymer is subjected to one or more chemical reaction that results in acid-functional groups being attached to the preliminary copolymer to form the acid-functional resin

A preferred method of making a resin (i) is aqueous suspension polymerization of a monomer mixture that contains vinyl aromatic monomer to make a preliminary polymer (i). Preferably, the monomer mixture contains monofunctional vinyl aromatic monomer in an amount, by weight based on the weight of the monomer mixture, 50% or more; more preferably 75% or more; more preferably 90% or more. Preferred monofunctional vinyl aromatic monomer is styrene. Preferably, the monomer mixture contains multifunctional vinyl aromatic monomer in an amount, by weight based on the weight of the monomer mixture, 50% or less; more preferably 25% or less; more preferably 10% or less. Preferably, the monomer mixture contains multifunctional vinyl aromatic monomer in an amount, by weight based on the weight of the monomer mixture, 0.5% or more; more preferably 1% or more; more preferably 2% or more. Preferred multifunctional vinyl aromatic monomer is divinylbenzene.

When making resin (i), preferably, preliminary polymer (i) is subjected to a chemical reaction with sulfuric acid to attach sulfonic acid groups to preliminary polymer (i) to produce resin (i). Preferably, in resin (i), the mole ratio of sulfonic acid groups to aromatic rings in resin (i) is 0.8:1 or more; more preferably 0.9:1 or more. Preferably, the mole ratio of sulfonic acid groups to aromatic rings in resin (i) is 2:1 or less.

A preferred method of making a resin (ii) is aqueous suspension polymerization of a monomer mixture that contains acrylic monomer to form a preliminary polymer (ii). Preferably, the monomer mixture contains monofunctional acrylic monomer in an amount, by weight based on the weight of the monomer mixture, 50% or more; more preferably 75% or more; more preferably 90% or more. Preferred acrylic monomers are unsubstituted-alkyl esters of acrylic acid, unsubstituted-alkyl esters of methacrylic acid, acrylonitrile, and methacrylonitrile; more preferred are methyl acrylate and acrylonitrile. Preferably, the monomer mixture contains multifunctional vinyl monomer in an amount, by weight based on the weight of the monomer mixture, 50% or less; more preferably 25% or less; more preferably 10% or less. Preferably, the monomer mixture contains multifunctional vinyl aromatic monomer in an amount, by weight based on the weight of the monomer mixture, 0.5% or more; more preferably 1% or more; more preferably 2% or more. Preferred multifunctional vinyl monomer are multifunctional vinyl aromatic monomers; more preferred is divinylbenzene.

When making resin (ii), preferably preliminary polymer (ii) is subjected to a chemical reaction to result in carboxylic acid groups being attached to preliminary polymer (ii) to form resin (ii). Preferably, in resin (ii), the mole ratio of carboxylic acid groups to polymerized units of monofunctional acrylic monomer is 0.8:1 or higher; more preferably 0.9:1 or higher. Preferably, the mole ratio of carboxylic acid groups to polymerized units of monofunctional acrylic monomer is 1.1:1 or lower.

The strength of the acidity of the acid-functional resin may be characterized herein by the pKa of an effective acid-functional monomer. The effective acid-functional monomer is determined by considering the acid-functional resin, then examining a polymerized unit that has an acid-functional group, then determining the polymerization bond that links that polymerized unit to other polymerized unit, then envisioning a monomer that would be present if that polymerization bond were to be reversed, and then determining the pKa of that monomer. For example, it is possible to imagine a hypothetical resin (i) that was made by first making a preliminary copolymer of styrene and divinylbenzene and then reacting the preliminary copolymer with sulfuric acid to make a resin that had one sulfonic acid group per aromatic ring. Then the effective acid-functional monomer would be styrenesulfonic acid, which has pKa of −0.53. For another example, it is possible to imagine a hypothetical resin (ii) that was made by first making a preliminary copolymer of methyl acrylate and divinylbenzene, and then reacting the copolymer with caustic to make a resin that had one carboxylic acid group per polymerized unit of methyl acrylate. Then the effective acid-functional monomer would be acrylic acid, which has pKa of 4.25.

Preferably, the acid-functional resin has pKa, as characterized by the pKa of the effective acid-functional monomer, of −4 or higher; more preferably −2 or higher; more preferably 0 or higher; more preferably 2 or higher; more preferably 3 or higher. Preferably, the acid-functional resin has pKa, as characterized by the pKa of the effective acid-functional monomer, of 8 or lower; more preferably 6 or lower.

Preferred acid-functional resins are amorphous. Preferred acid-functional resins are crosslinked.

Preferably the collection of beads has harmonic mean size of 200 nm or higher; more preferably 300 μm or higher; more preferably 500 μm or higher. Preferably the collection of beads has harmonic mean size of 1500 μm or lower; more preferably 1000 μm or lower.

The beads may be characterized by their tendency to swell when submerged in phenol at 23° C. It is noted that, in general, beads that are made of crosslinked resin often are capable of swelling when submerged in a liquid. Preferably, the beads used in the present invention will increase their volume by 20% or more when submerged in phenol at 23° C.

Preferably, the beads contain one or more metal selected from palladium, platinum, silver, gold, rhodium, ruthenium, copper, iridium, and mixtures thereof; more preferably selected from palladium, platinum, or a mixture thereof; more preferably palladium. Preferably, the mole % of the metal that is in the zero-valence state is 80% or more; more preferably 90% or more. Preferably, the metal is present in the beads in the form of crystals. Preferably, the harmonic mean diameter of the crystals is 10 μm or smaller; more preferably 3 μm or smaller; more preferably 1 μm or smaller.

The concentration of metal in the beads may be characterized by the ratio (“M2B”) of the weight of metal to the volume of the collection of beads. Preferably, that ratio M2B is 0.5 g/L or higher; more preferably 1 g/L or higher; more preferably 2 g/L or higher. Preferably, that ratio M2B is 10 g/L or lower; more preferably 5 g/L or lower.

The reaction mixture optionally contains one or more additional ingredients. Preferred additional ingredients include solvents that do not undergo chemical reaction under the conditions of the hydrogenation reaction. Preferred solvents are hydrocarbons that are liquid at 23° C. under 1 atmosphere of pressure. Preferred solvents are hydrocarbons having 6 or more carbon atoms. Preferred solvents are hydrocarbons having 12 or fewer carbon atoms; more preferably 10 or fewer carbon atoms. Preferably the sum of the masses of reactant (a), solvent, catalyst, and hydrogen, as a percentage of the total mass of the reaction mixture, is 50% or more; more preferably 75% or more; more preferably 90% or more; more preferably 95% or more

The metal may be introduced into the beads by any method. In a preferred method, acid-functional resin is brought into contact with a solution of a soluble salt of a cation of the desired metal and an anion in a solvent. During this contact, some or all of the labile hydrogen atoms in the acid-functional groups on the resin are considered to exchange with cations of the desired metal. Then, after removal of the solvent and optional additional steps, the cations of the desired metal are crystals of zero-valent metal. A preferred method of introducing metal into the beads is described in U.S. Pat. No. 8,552,223.

The reaction mixture may be formed by any method. The ingredients (reactant (a), hydrogen, catalyst, and any additional optional ingredients) may be brought together in any order in any combination. Preferably, a preliminary mixture that contains reactant (a), hydrocarbon solvent, and catalyst is formed in a vessel, the vessel is sealed, and then hydrogen gas in introduced into the vessel, thus bringing the hydrogen gas into contact with that preliminary mixture to form the reaction mixture.

To conduct the hydrogenation reaction of the present invention, preferably the reaction mixture is subjected to pressure of 2 bar or higher; more preferably 5 bar or higher; more preferably 10 bar or higher; more preferably 18 bar or higher. To conduct the hydrogenation reaction of the present invention, preferably the reaction mixture is subjected to pressure of 30 bar or lower.

To conduct the hydrogenation reaction of the present invention, preferably the reaction mixture is subjected to temperature of 60° C. or higher; more preferably 80° C. or higher; more preferably 100° C. or higher. Preferably, during the method of the present invention, the average temperature of the reaction mixture does not ever rise above temperature TMAX, where TMAX is preferably 200° C. or lower; more preferably 180° C. or lower; more preferably 160° C. or lower; more preferably 140° C. or lower.

Preferably, the reaction mixture is held at a temperature of 60° C. or higher and pressure of 2 bar or higher for a time period of 1 hour or more; more preferably 2 hours or more. Preferably, the reaction mixture is held at a temperature of 60° C. or higher and pressure of 2 bar or higher for a time period of 12 hours or less; more preferably 9 hours or less; more preferably 6 hours or less.

Preferably, the reaction mixture is subjected to agitation, preferably by operation of a mechanical rotary stirring device within the reaction mixture. The rotary stirring device may be powered by any method, including, for example, rotary force applied by contact with a rotating element such as a drive shaft, or rotary force applied by a rotating magnetic field.

It is contemplated that one advantage of the present invention is that the beads have good mechanical stability. For example, the beads preferably do not change significantly during the agitation of the reaction mixture. The change, if any, in the beads can be assessed by measuring the harmonic mean diameter of the beads, measured after drying and removing solvent from the beads, before and after agitation. Preferably the ratio of the harmonic mean diameter after agitation to the harmonic mean diameter before agitation is from 0.9:1 to 1.05:1.

The following are examples of the present invention.

In the following examples, “conversion” measures how much of the phenol was consumed, expressed as a percentage:

conversion=100*(1−([final amount of phenol]/[initial amount of phenol])).

The term “selectivity” describes how much of the desired product (cyclohexanone) was formed as compared to unwanted products, expressed as a percentage:

selectivity=100*[C]/([C]+[D]),

where [C] is the amount of cylohexanone produced, and [D] is the sum of the amounts of all other products of the chemical reactions that take place during the hydrogenation process.

The resins used in the following examples were these. All resins were obtained from the Dow Chemical Company.

| | | Acid- | Polymer | Harmonic |
|---|---|---|---|---|
| | | Functional | Compo- | Mean |
| Label | Type | Group | sition | Diameter (μm) |
| Resin A15 | AMBERLYST ™ 15 | sulfonic | vinyl | 600-850 |
| | | | aromatic | |
| Resin A36 | AMBERLYST ™ 36 | sulfonic | vinyl | 600-850 |
| | | | aromatic | |
| Resin A35 | AMBERLYST ™ 35 | sulfonic | vinyl | 700-950 |
| | | | aromatic | |
| Resin HP | IMAC ™ HP333 | carboxylic | acrylic | 500-700 |

### COMPARATIVE EXAMPLE 1C

To a 15 mL glass-lined, steel, pressure reactor (Endeavor™ Reactor available from Argonaut Technologies) equipped with mechanical stirring and gas inlet was added strong acid cation exchange resin (0.9 g). This resin was conditioned by rinsing the resin three times with 5 mL aliquots of acetone. After the resin had been conditioned, the reactor was charged with phenol (2.0 g) and isooctane solvent (3.5 g). Once all the ingredients were added, the reactor was closed, stirring commenced (350 rpm) and inertion process commenced by pressurizing to 21 bar (300 psi) with N₂ (inert gas) followed by depressurization. This pressurization/depressurization cycle was done a further two times. Upon completion of reactor inertion, the reactor contents were pressurized to 21 bar (300 psi) with H₂(g). The reactor was then heated to 110° C. for four hours. After the four hours, the contents of the reactor were allowed to cool, and the liquid contents were subjected to gas chromatography/mass spectrometry (GC/MS) to determine phenol conversion and cyclohexanone selectivity. Results are listed in Table 1.

### EXAMPLES 2-7

To a 15 mL glass-lined, steel, pressure reactor (Endeavor™ Reactor available from Argonaut Technologies) equipped with mechanical stirring and gas inlet was added metal doped polymer catalyst (prepared as described in Example 2 of U.S. Pat. No. 8,552,223B2) (varying amounts, as shown in Table 1). This catalyst was conditioned by rinsing the catalyst three times with 5 mL aliquots of acetone. After the resin had been conditioned, the reactor was charged with phenol (2.0 g) and isooctane solvent (3.5 g). Once all the ingredients were added, the reactor was closed, stirring commenced (350 rpm), and inertion process commenced by pressurizing to 21 bar (300 psi) with N₂ (inert gas) followed by depressurization. This pressurization/depressurization cycle was done a further two times. Upon completion of reactor inertion, the reactor contents were pressurized to 21 bar (300 psi) with H₂(g). The reactor was then heated to 110° C. for four hours. After the four hours, the contents of the reactor were allowed to cool, and the liquid contents were subjected to GC/MS to determine phenol conversion and cyclohexanone selectivity. Results are listed in Table 1.

Results:

TABLE 1
All of Examples 2-7 used 2.8 grams of metal per liter of resin.

| | | | amount of | Conversion | Selectivity |
|---|---|---|---|---|---|
| Example | Metal | Resin | catalyst | (%) | (%) |
| 1C | none | Resin A15 | 0.9 g | 0 | 0 |
| 2 | Pd | Resin A15 | 0.9 g | 69 | 86.7 |
| 3 | Pd | Resin A36 | 0.9 g | 81 | 93.6 |
| 4 | Ru | Resin A36 | 0.9 g | 20 | 0 |
| 5 | Pd | Resin A35 | 0.09 g | 78 | 63.3 |
| 6 | Pd | Resin A35 | 0.009 g | 63 | 55.0 |
| 7 | Pd | Resin HP333 | 0.9 g | 90 | 95.7 |

The inventive Examples 2-7 showed some conversion of reactant, while the comparative example 1C showed no conversion. Palladium performed better than ruthenium. Example 7, which used a weak-acid resin, showed the best conversion and the best selectivity.

1. A method of hydrogenation comprising forming a reaction mixture comprising
(a) one or more reactant selected from the group consisting of phenol, one or more derivatives of phenol, and mixtures thereof;
(b) hydrogen; and
(c) catalyst, wherein the catalyst comprises beads that comprise one or more acid-functional organic resin and one or more metal selected from the group consisting of palladium, platinum, silver, gold, rhodium, ruthenium, copper, iridium, and mixtures thereof.

2. The method of claim 1, wherein the acid-functional organic resin comprises carboxylic acid groups.

3. The method of claim 1, wherein the acid-functional organic resin comprises acrylic polymer.

4. The method of claim 1, wherein the metal comprises palladium.

5. The method of claim 1, wherein the reactant is phenol.

6. The method of claim 1, wherein the hydrogenation produces one or more products that comprise cyclohexanone or a derivative thereof.

7. The method of claim 1, wherein the method is conducted at temperature less than 200° C.
